(19) United States
(12) Patent Application Publication
(10) Pub. No.: US 2010/0120109 A1
(43) Pub. Date: May 13, 2010

(54) High dry matter sweet potatoes for fuel ethanol, green energy, chips and fries

(75) Inventor: Janice Ryan-Bohac, North Charleston, SC (US)

Correspondence Address: BARNES & THORNBURG LLP, P.O. BOX 2786, CHICAGO, IL, 60690-2786, US

(73) Assignee: CAROLINA ADVANCED RENEWABLE ENERGY, LLC, North Charleston, SC (US)

(21) Appl. No.: 12/590,789

(22) Filed: Nov. 13, 2009

Related U.S. Application Data
(60) Provisional application No. 61/114,358, filed on Nov. 13, 2008.

(51) Int. Cl. A01H 5/00; C07C 29/74; C12P 7/06; A23L 1/01; A01G 1/00

(52) U.S. Cl. 435/161; 800/298; 568/913; 426/441; 47/58.1 FV

(57) ABSTRACT

A variety type of sweetpotato, Ipomoea batatas, that was grown in such a manner to maximize the tonnage of storage roots produced. These types of sweetpotato can accumulate from 28% to 40% dry matter in the root which is mostly carbohydrate. By combining high levels of carbohydrate (mostly starch and some sugars) with high tonnage of roots per acre, this type of sweetpotato produces some of the highest levels of ethanol per acre found in any temperate crop. This type of sweetpotato with its uniquely high dry matter, high starch content, and mealy texture can also be used to produce sweetpotato chips and fries that have significantly less oil than commercially available low dry matter sweetpotato varieties and have an excellent, distinctive, texture and flavor.

This application claims priority to copending U.S. provisional application Ser. No. 61/114,358, filed Nov. 13, 2008.

Sweetpotatoes characterized by high dry matter and high starch content, produce improved yield of fuel ethanol, improved and distinctive chips and fries, and are a source of green energy.

### BACKGROUND

Although there a large number of sweetpotato varieties with a wide range of appearance, growth parameters, and uses, not a lot is known about the genetics of this crop species. Sweetpotato is the 7^{th} largest food crop in the world and is a major food staple in many countries, mostly in the tropics.

Sweetpotatoes are underground storage roots, not tubers. The botanical description of a tuber is a short, thickened portion of an underground stem. A tuber has eyes composed of a ridge bearing a ‘scale-like’ leaf with tiny meristematic buds in the axial of this scale-like leaf. In contrast, a sweetpotato is a true root, not an underground tuber that has buds. The anatomy of a storage root is the same as any root, with an additional ability to expand radially to store additional starch and nutrients. The sweetpotato crop is asexually propagated crop from ‘seed’ roots and plant cuttings.

Currently grown U.S. sweetpotato varieties were developed to be eaten as a highly nutritious and delectable vegetable. This variety type has been bred for specific traits for that use. They produce relatively uniform, attractively shaped storage roots, with dark orange flesh, a sweet, delicious flavor, and a moist texture (from 77% to 81% moisture). The typical weight of these roots is from 6 to 16 ounces. The typical fresh weight yields of these varieties will range from 12 to 15 tons of marketable roots per acre. Other countries have other varieties of sweetpotatoes.

Fuel ethanol has been produced from various plant sources and countries like Brazil have become energy independent based on ethanol produced from sugar cane and cassaya. In the US, some feedstock crops like corn, can successfully produce ethanol and the majority of the fuel ethanol plants are located in the corn belt—the midwestern states. However, the relatively low yield of ethanol (about 300 gallons per acre) and high inputs of water and fertilizer limit the ability of corn to replace a significant amount of gasoline in the US. Although the southern states have many advantages for growing ethanol crops, such as a longer growing season, lower land prices, available labor, etc. almost no fuel ethanol plants have been built there. The high summer temperatures and poor soils in the southern states means that corn yields are less than half the yields in the midwestern states. The number one obstacle to locating this industry in the southern states is the identification of a high yielding, well adapted feedstock crop.

Sweetpotato chips and fries are available from sweet dark orange vegetable varieties, but these unsuitable varieties make chips that are high in fat, dark and often burnt, with variable texture and poor shelf life.

### SUMMARY

A variety type of sweetpotato, Ipomoea batatas, were grown in such a manner to maximize the tonnage of storage roots produced. These types of sweetpotato can accumulate from 28% to 40% dry matter in the root which is mostly carbohydrate. By combining high levels of carbohydrate (mostly starch and some sugars) with high tonnage of roots per acre, this type of sweetpotato produces some of the highest levels of ethanol per acre found in any temperate crop. This type of sweetpotato with its uniquely high dry matter, high starch content, and mealy texture can also be used to produce sweetpotato chips and fries that have significantly less oil than commercially available low dry matter sweetpotato varieties and have an excellent, distinctive, texture and flavor.

A horticultural method to maximize yield of high dry matter type sweetpotatoes, includes the step of:
(a) transplanting 10 inch long cuttings with a Holland mechanical pocket transplanter 12 inches apart in plant beds 38 inches apart;
(b) watering the plants, e.g., by the transplanter and natural rainfall;
(c) planting a crop in the middle of a month with a climate essentially the same as the average climate mid-May in South Carolina;
(d) harvesting the crop at the last frost at approximately 150-160 days after planting; and
(e) fertilizing the crop with nitrogen, phosphorus, potash, sulfur, boron, manganese and copper wherein fertilizing comprises approximately 45 pounds of nitrogen, approximately 110 pounds of phosphorus and potash, approximately 5 pounds of sulfur, approximately 0.4 pounds boron, approximately 10 pounds of manganese, and approximately 0.1-0.2 pounds of copper per acre.

A suitable sweetpotato is from the variety CX-1.

A method to produce fuel ethanol includes the steps of:
(a) obtaining sweetpotato roots characterized by high dry matter preferably about 28-40%;
(b) producing fuel ethanol from the sweetpotatoes.

Suitable sweetpotato roots are from the variety designated CX-1. the method can be used on a commercial scale or in a laboratory.

A laboratory method to produce fuel ethanol includes the steps of:
(a) peeling, grating and drying high dry matter type sweetpotato roots;
(b) placing the dried sweetpotato roots in flasks and adding water to reconstitute them;
(c) adjusting ph to 6.5 and adding amylase to the reconstituted composition and heating the results to boiling with stirring;
(d) cooling, adjusting pH to 4.5-5.0, e.g., with concentrated sulfuric acid;
(e) adding gluco-amylase, yeast and water; and
(f) incubating preferably for approximately 42 hours.

A commercial method to produce fuel ethanol includes the steps of:
(a) chopping the sweetpotato roots with an industrial auger;
(b) processing fragments of roots into a slurry;
(c) adding amylase and heating to boiling with stirring and boiling for about 90 minutes;
(d) cooling to 50° C., adjusting pH to 4.5-5.0, e.g., with concentrated sulfuric acid;
(e) adding gluco-amylase, yeast and water;
(f) incubating in fermentation tank, e.g., for about 3-6 hours;
(g) removing ethanol and water, separating the ethanol by fractional distillation; and
(h) removing final water from ethanol by molecular sieves.

The slurry is produced e.g., by passing it through a continuous double helix blender while adding a minimal amount of water.

A method to produce sweetpotato chips and fries includes the steps of:
(a) obtaining high dry matter sweet potatoes;
(b) washing, peeling and slicing the sweetpotatoes;
(c) for french fries cutting circular slices of about 30 mm into strips, for chips cutting about 5 mm circular cross sections;
(d) pre-treating fries and chip slices in e.g., 15% saline for 10 minutes and drying; and
(e) cooking the cut pieces in hot oil until the chips or fries float and the water is cooked off.

A suitable sweetpotato clone is characterized by 28-40% high dry weight. An embodiment is CX-1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a photograph of a flat of rose-skinned, large roots from CX-1 sweetpotatoes. P FIG. 2 is a photograph of a very large (17 lbs.) “turnip” shaped CX-1 sweetpotato and an 8 ounce copper-skinned “Beauregard” sweetpotato (accounts for 90% of U.S. commercial market).

FIG. 3 are photographs of (A) the upper side of a CX-1 leaf; and (B) the under side of a CX-1 leaf.

FIG. 4 is a photograph looking down on a planting of CX-1 sweetpotatoes.

FIG. 5 is a photograph of chips made from CX-1 sweetpotatoes.

### DETAILED DESCRIPTION

Types of sweetpotatoes with high percent of dry matter and high starch content, provide improved biofuel and food. As an example, sweetpotato roots of CX-1 were used to produce fuel ethanol. Estimates of at least 1500 gallons per acre of sweetpotatoes were based on field tonnage per acre, and on laboratory tests of the amount of ethanol produced per ton of dry matter from storage roots. Sweetpotato roots of CX-1 and similar dry, starchy variety types were also used to make chips and fries. These types make excellent chips and fries compared to what can be made from the very sweet, watery texture found in vegetable types of sweetpotato like the leading U.S. variety, Beauregard. The dry matter content of these dry types is 28% and higher. In contrast to vegetable type sweetpotatoes, the dry matter content ranges from 19 to 25%. The texture of the CX-1 and other dry sweetpotato types was dry and mealy in contrast to vegetable types that are moist and sweet with visible syrup in the flesh. The chips made from the dry types were crisp, delicious, and light in color, similar to a commercial chip made from the white potato. Both the CX-1 chips and fries with high dry matter take up significantly less oil and were less bitter than these products made from the white potato.

The sweetpotato varieties disclosed herein include those that have been genetically modified e.g., increased pathogen resistance, pest resistance, drought tolerance, and stress tolerance. Such engineered varieties may also include genetic elements to increase one or more components for enhanced biofuel production, such as for example, increased dry matter and increased starch material.

In some aspects, the sweetpotato varieties disclosed herein include for example, more than 28% dry matter, more than about 29% dry matter, about 28-40%, about 30-45%, on an average about 30%-35%, about 32%, about 34%, and about 35%. In some aspects, the sweetpotato varieties disclosed herein include for example, at least 28%, or 29%, or 30%, or 31%, or 32%, or 33%, or 34%, or 35%, or 36%, or 37%, or 38%, or 39%, or 40% and up to 50% dry matter.

In some aspects, the sweetpotato varieties disclosed herein include for example, more than about 60% starch, 65% starch, 70% starch, 75% starch, 78% starch, 80% starch, and 85% starch in the dry matter by weight. In some aspects, the starch content ranges from about 70% to about 85% when measured in the dry matter content. In some aspects, the starch content ranges within an average range of about 50% to about 85% of the dry matter.

### Example 1

### Production of CX-1 Variety

The sweetpotato variety Xushu 18 was a variety that was publicly released in 1972 from China. It was bred by researchers from the Jiangsu Academy of Sciences in Jiangsu Province, China. Xushu 18 was a seedling from a cross from the variety Xindazi and breeding line 52-45. (Gitoner, C. 1996. Potato and Sweetpotato in China: systems, constraints, and potential. International Potato center. Lima, Peru.).

Xushu 18 has been a popular variety in China, a country that is a major producer of sweetpotatoes. Researchers in China report that during several decades, dozens of new varieties derived by mutation from named varieties have been registered. The Xushu sweetpotato Research Center in China reports that a single selection of Xushu 18 with superior characteristics was named Xu 77-6 and was selected to replace normal Xushu 18.

Xushu 18 was not developed as a table vegetable. It was developed for its production of starch and for animal feed. It is not sweet or delicious, but is white fleshed, very bland and very dry, about 28% dry matter. Commercial yields in China have been reported from 6.88 ton up to 18.5 tons of fresh root weight per acre.

Starting with the Xushu 18, a distinctive, new clone was selected and developed for large roots, very high dry matter and higher total yield. The original Xushu 18 clone was obtained from the Plant Genetic Resourses Unit, USDA, ARS, Griffin, Ga. After years of field trials, it was determined that a selected mutant clone, CX-1 produced with a specific growing method had superior yield and higher dry matter content compared to published data on Xushu 18 (Table 1).

Samples of CX-1 will be deposited and maintained by the Foundation Plant Services, University of California, Davis, Calif.

The color analysis of CX-1 is: Netscape colors in Hue Order—Skin color is Pale Violet Red (RGB: 219, 112, 147; HSV: 340.4, 0.489, 0.859). Flesh color is Lemon Chiffon (RGB: 255, 250, 205; HSV: 54.0, 0.196, 1.000). Reference: www.efg2.com/Lab/Graphics/Colors/ColorCharts.htm.

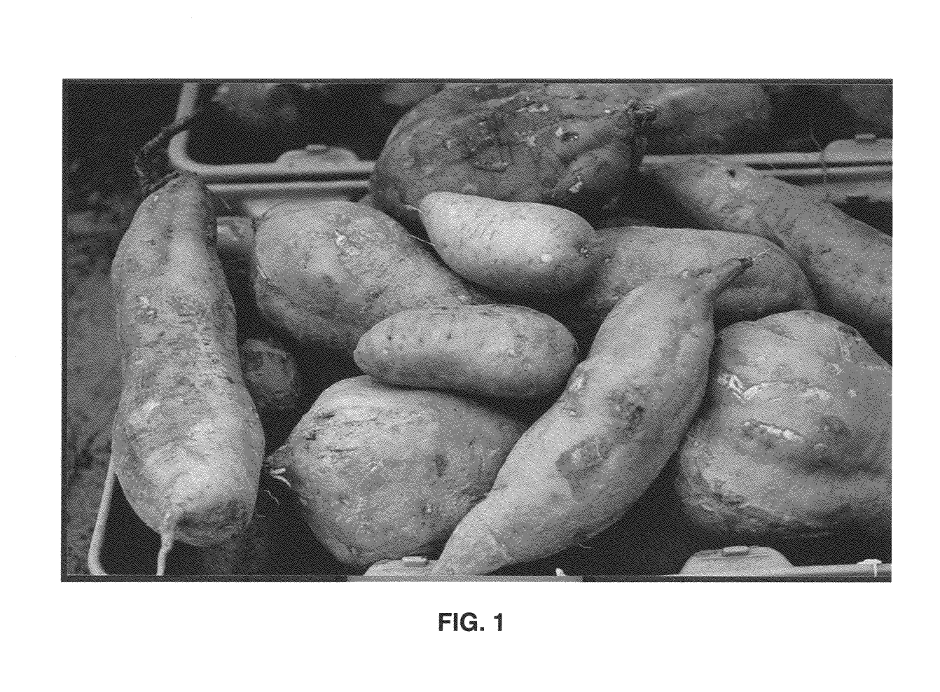

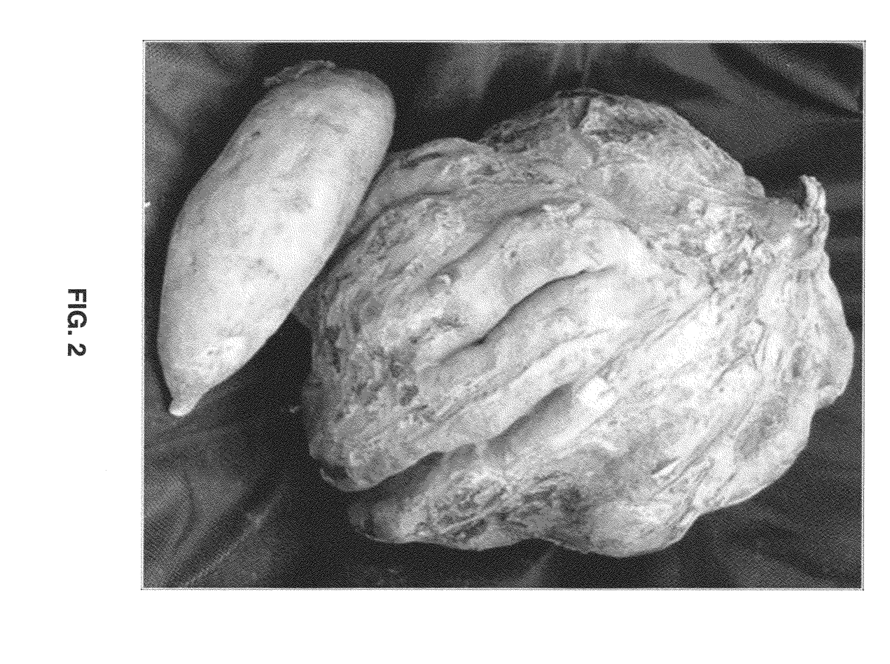

CX-1 produces large to very large storage roots from 1 pound up to 17 lbs. The storage roots initiate at about 45 days after planting. The large roots have a smooth, oblong, blocky shape (FIG. 1). The very large roots are deeply grooved with a ‘turnip’ type shape (FIG. 2). The storage roots have a medium rose-hued skin that is smooth and attractive on the smaller roots and is rougher and non-uniform on the very large roots. The internal flesh is a light yellow with a uniform, starchy appearance.

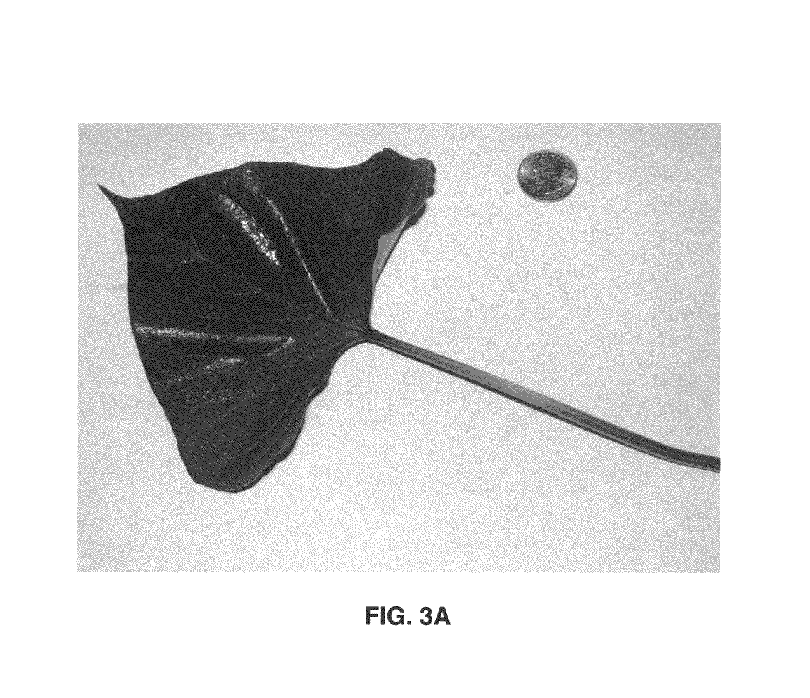

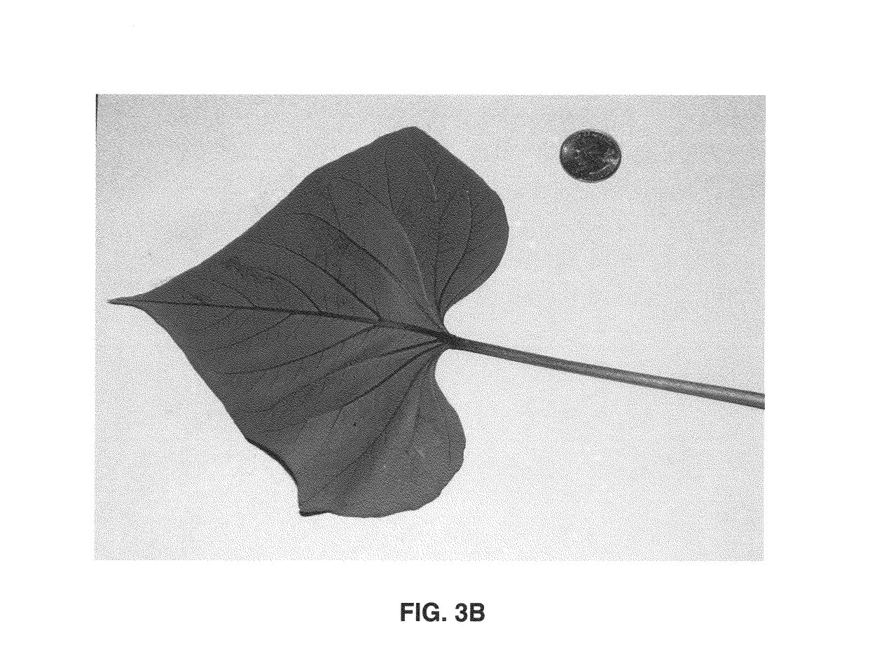

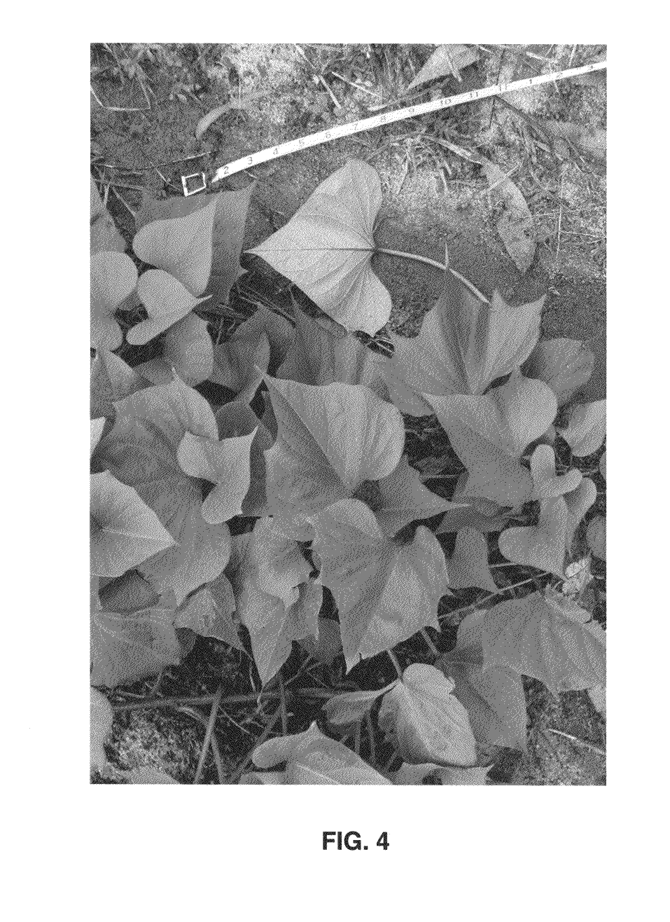

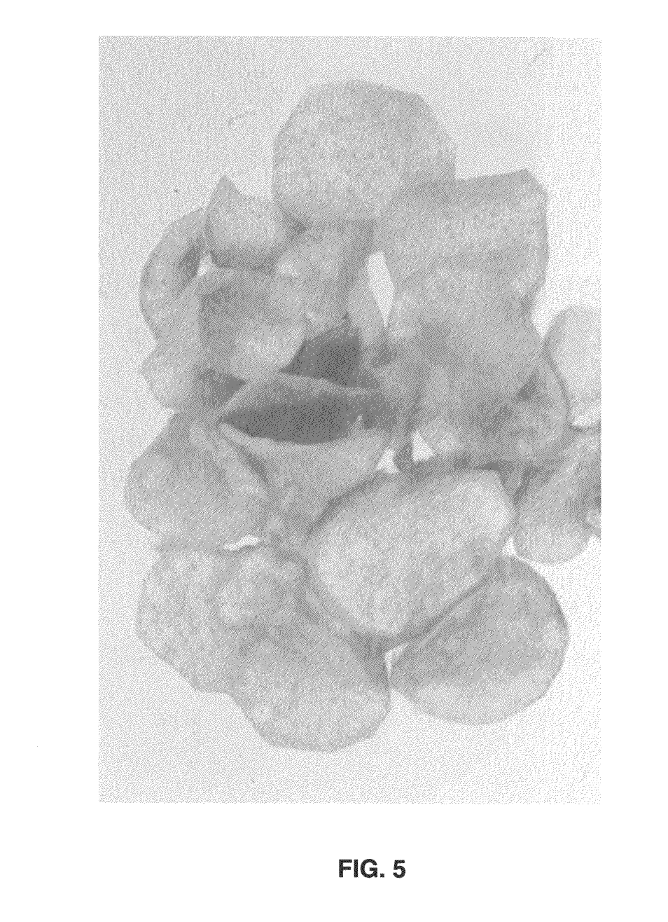

The leaves of the CX-1 are a medium green, cordate-shaped leaf with a purple juncture where the base of the leaf blade meets the petiole and the veins are often purple from the juncture grading to green at the leaf outer edges (FIGS. 3A and 3B). The cultivar makes a vigorous vine longer and more robust than Beauregard, the leading US sweetpotato variety. (FIG. 4).

### Example 2

### Horticulture Methods to Maximize Yield

Horticultural methods were developed that differed from standard sweetpotato methods, in order to maximize the yield of CX-1. The standard spacing for vegetable sweetpotato plants is 9 inches apart in the beds, the spacing developed for CX-1 was 12 inches apart, which allowed the roots to grow larger. A Holland-type pocket transplanter was used to plant cuttings approximately 10 inches in length. All plots were watered through the transplanter, and occasionally supplemented with irrigation the first 30 days. The remainder of the season, the crop received only normal rain. In order to maximize yield, the CX-1 plants were transplanted in late April or May and allowed to grow until the vines were killed by frost. The vegetable sweetpotatoes are harvested at 90 to 100 days after planting, the CX-1 are harvested 150 to 160 days after planting (or until the first major frost).

Soil samples were submitted for soil testing. In all trials run in South Carolina and Florida, it was found that the CX-1 performed well on sandy loam soils that were low in organic matter. It is expected that the variety would perform well throughout the Coastal Plain soils that are found from southern New Jersey to Florida, and the southern counties in Alabama, Mississippi, Louisiana and East Texas. Soil tests for sweetpotato production in this type of soil recommend 90 lbs of Nitrogen, 110 lbs of Phosphorus and 110 lbs of Potash per acre. It was discovered that the highest yield of CX-1 used half of the recommended Nitrogen —45 lbs of Nitrogen per acre. Also added was recommended rate of 110 lbs per acre of Phosphorus and Potash combined with micronutrients—5 lbs of sulfur, 0.38 lbs of Boron, 10 lbs of Manganese and 0.13 lbs of Copper per acre. Also used was 1.5 pints per acre of the herbicide Dual and the grass herbicide Poast at 1 pint per acre. The insecticides sprayed were Malathion and Thionex as needed. No fungicides or nematacides were required.

Field trials were run in North Florida and in South Carolina. Storage roots of CX-1 contained 32% dry matter. The yield at 120 and 150 days after planting was found to be 12 tons to 16 tons of dry matter per acre, respectively. Samples of the sweetpotatoes were sent to the TVA laboratory in Muscle Shoals, Ala. for a chemical analysis. It was found that the CX-1 roots were approximately 32% dry matter. This dry matter was found to be 74% starch, which is readily converted to fuel ethanol in the process. Two commercially important traits make CX-1 superior to US varieties for fuel ethanol and clearly differentiate CX-1 as a new variety derived from Xushu 18. One key fact that indicates CX-1 is a stable mutant of Xushu 18, is that CX-1 roots have dramatically higher dry matter 32%. In contrast the parent clone, Xushu 18, has 27-29% dry matter. Also yield is higher in CX-1—fresh weight ranges from 37.5 to 49 tons per acre and (dry weight from 12 to 16 tons per acre). Published fresh weight data for Xushu 18 from China, Peru and Thailand have average values of 17.8, 13.6, and 12.0 tons/acre, respectively. (Table 1).

Samples of the CX-1 roots were subjected to a chemical analysis and the data is presented in Table 2. The typical vegetable type sweetpotato is very high in water and sugar lower starch content). In contrast, the CX-1 has higher starch content and lower water content.

Laboratory tests have determined that CX-1 will produce 121 gallons of ethanol per dry ton of sweetpotato roots. Under growing methods employed in South Carolina and Florida 12 to 16 tons of storage root dry matter per acre was produced. Using these methods, CX-1 can produce from 1450 to 1940 gallons per acre.

The ethanol is produced by yeast fermentation of the cooked storage roots by methods known to those of skill in the art.

### Example 3

### A Laboratory Method to Produce Ethanol

(a) To produce ethanol from sweetpotato roots, the roots are harvested, cleaned, and cooked. The sweetpotatoes were peeled and grated into small pieces (¼ ″×⅛″×1″). Moisture content was determined by placing about 3 g of grated feedstock in two OHaus moisture meters for 90 min at 110° C. The moisture content was averaged and determined to be 32.5% for CX-1. Fresh samples can also be weighed and dried in an oven at 150 F for 3-4 days. The dry matter is percentage of material weight of the original fresh weight samples.

(b) All reactions were conducted in a 250 ml flask. About 11-12 g of the dried sweetpotato roots was placed in flasks and water added to reconstitute to 15% solids content. The pH was checked and adjusted to about 6.5. Amylase was added (1 ml). The mixture was heated to boiling and boiled for 90 minutes with constant stirring with a magnetic stir bar.

(c) After cooling to 50° C., the mixture adjusted to pH 4.5-5.0 with concentrated sulfuric acid. Gluco-amylase (1 ml) was added and the mixture was reacted with water at 50° C. for 30 min. Yeast (1 mg) was added after cooling to 35° C. The flask was fitted with a water-sealed top and incubated at 30° C. for up to 45 hours. Each test was conducted in duplicate.

(d) Samples were taken at the beginning of fermentation, and then at about 15 hours, 24 hours, and 40 hours. Samples were analyzed for sugars and ethanol. The ethanol yield was calculated as g of ethanol per 100 g of dry sweetpotato.

(e) CX-1 yielded 33.5-38.0 g ethanol/100 g DM at 15 hours with a final ethanol yield of 40 g/100 g DM at 42 hours. In the final analysis, CX-1 produced 121 gallons of ethanol per dry ton of sweetpotato. Based on the yield in North Florida (150 days of growth) of 16 tons of dry matter per acre, the CX-1 can produce up to 1936 gallons of ethanol per acre. Based on a yield of 12.8 tons of dry matter per acre in South Carolina (120 days of growth), CX-1 can produce up to 1548 gallons of ethanol per acre.

### Example 4

### Comparison Between Commercial Production of Fuel Ethanol from High Dry Matter Sweetpotatoes to Corn

The general commercial process for producing fuel-ethanol from any starch crop is to convert starch to simple sugars and then ferment the sugars to produce ethanol. Distillation is used to separate the ethanol from the water and other components of the fermented stream. The last water is removed from the ethanol by such unit operations as molecular sieves, and the ethanol combined with gasoline to produce fuel-ethanol. Methods that maximize yield and minimize costs of producing fuel ethanol have been under development since Henry Ford designed the original Model T Ford to run on corn ethanol. It is apparent that these methods are economically successful, since the majority of the 9 billion gallons of fuel ethanol produced in the US in 2008 was derived from corn at prices competitive with gasoline.

Although both sweetpotato and corn are carbohydrate crops, there are some significant differences in starch structure, sugars, and other biochemical and textural differences that will require specific technology to maximize the commercial yield of fuel ethanol from these type of sweetpotatoes.

The key differentiating points between the process of producing fuel ethanol from sweetpotato compared to corn are: a) the process to convert whole sweetpotato into slurry, b) identification of the specific enzymes to optimize the conversion of sweetpotato starch into simple sugars on a commercial scale, considering the endogenous enzymes and possible inhibitors, c) identification of the specific strain(s) of yeast to ferment the sweetpotato process, and d) the sweetpotato's high water content that minimizes the large amounts of local water needed for processing dry corn.

The sweetpotato roots require different processing than corn. In the corn process, the corn kernels are ground in a mill, and the ground corn is mixed with hot water and enzymes to dissolve the starch at low viscosity. In contrast, the physical process of converting large, very dense and hard roots of this type of sweetpotato into a slurry is more difficult than the easy process of grinding corn into cornmeal. A large industrial auger must be used to grind up the dense sweetpotato flesh. The large pieces are then processed into a slurry by passing through a continuous double helix blender while adding a minimal amount of water and enzyme. The output from the blender is slurry of water, starch, and other insolubles from the sweetpotato.

This slurry is transferred to fermentation tanks and temperature taken up to boiling at 100° C. for 90 minutes, cooled to 50° C., and the pH adjusted 4.5-5.0 with concentrated sulfuric acid. Gluco-amylase and yeast are added, and the mash is fermented approximately 42 hours to maximize ethanol yield. The water/ethanol mixture is removed and subjected to fractional distillation. The residual ethanol (about 10%) is removed with microfiltration and fuel ethanol can then be blended with gasoline to make E-10 or E-85.

Over a hundred years of research have been conducted to optimize the conversion of corn into fuel ethanol. Laboratory studies in conjunction with the present disclosure showed that ethanol can be produced from the CX-1 type of sweetpotatoes using the same enzymes used in the corn process to reduce the viscosity of the sweetpotato starch slurry and other enzymes to break the sweetpotato starch into simple sugars. However, sweetpotato starch molecules from varieties like CX-1 are different is size and branching than corn starch. Researchers report that raw sweetpotato starch is more resistant, than raw corn starch is to digestive enzymes like amylases. Ethanol yield from these type of sweetpotatoes will only be maximized by testing specific enzymes and reaction conditions that maximize the breakdown of the sweetpotato starch molecules. In the laboratory it was determined that by boiling the sweetpotato slurry for 90 minutes, the starch was more hydrolysable by the amylases.

Another difference between the ethanol process for sweetpotato compared to corn is the need to minimize side-reactions such as production of hydroxymethylfurfural which can inhibit growth and alcoholic fermentation by yeast. Sweetpotatoes also contain endogenous enzymes that could hinder or enhance the process depending on the specific enzymes added, cofactors, and temperature, pH and other physical conditions. Specific yeast strains for this type of sweetpotato that increase the yield of alcohol in the process, or reduce temperature or time of fermentation are identified by routine experimentation using methods disclosed therein.

One major advantage of processing sweetpotato over corn is that laboratory data shows that the sweetpotato process requires significantly less water added compared to the process using dry cornmeal. A sweetpotato such as CX-1, with 68% moisture needs very little added water. In contrast, corn ethanol biorefineries need to be located near a large source of water due to the large amounts of water needed to process the corn.

### Example 5

### A Method to Produce Sweetpotato Chips and Fries

(a) The high dry matter sweetpotatoes (CX-1) were washed and peeled. For chips, the sweetpotato was sliced into cross sections using a standard Berkel meat slicer used in commercial kitchens. The slices were 5 mm thick. The slices were subjected to pre-treatment by soaking in warm 15% saline for 10 minutes. The slices were dried on paper towels or dried with warm air, prior to cooking.

(b) A small standard electric French fry cooker with baskets was used to cook the chips. Canola oil was preheated to 325° F. Sweetpotato slices were placed in the baskets and dropped in the hot oil. Slices were cooked for 3-4 minutes until the chips floated and the water had cooked off observed by cessation of bubbles. Slices were cooled on paper towels and were ready for packaging.

(c) For French fries, the sweetpotatoes were washed and peeled. The sweetpotato was sliced into cross sections using a standard Beckel meat slicer used in commercial kitchens. The circular slices were 30 mm thick. The circular slices were then cut into strips for French fries approximately 30 mm wide. The fries were not subjected to soaking in warm 15% saline and dried as a pre-treatment.

(d) A small standard electric French fry cooker with baskets was used to cook the fries. Canola oil was preheated to 325° F. Sweetpotato fries were placed in the baskets and dropped in the hot oil. Slices were cooked for 6 minutes until the fries floated and the water had cooked off—observed by cessation of bubbles. Fries were cooled on paper towels and were ready for serving or freezing.

TABLE 1

| | | | Fresh Weight in | Fresh Weight in | Root Dry |
|---|---|---|---|---|---|
| Researcher | Location | Status | Tons/ha | Tons/A | Matter % |
| Ma Daifu, CIP | China (mean | Virus Free | 40 | 17.8 | 29 |
| | 19 locations)^{u} | Virus Check | 34 | 15.2 | 28 |
| Gruneberg, Crop Sci | Peru | Mean | 30 | 13.6 | 25 |
| | Tingo Maria | Highest | 70 | 31.5 | NR^{a} |
| | La Molina | Lowest | 5 | 2.2 | NR |
| Xie Yi Zhi, ARC | Thailand | Highest | 27 | 12.0 | 26 |
| Ryan-Bohac | USA | FL | | 49.0 | 32 |
| CAREnergy | USA | SC | | 37.5 | 32 |

^{a}NR is Not Reported; ha = hectors; A = acres
Wolfgang, G., Manrique, K., Zhang, D, and Hermann, M. 2005. Genotype X Environment interactions for a diverse set of sweetpotato clones evaluated across varying ecogeographic conditions in Peru. Crop Science. 45: 2160-2171.
Zhi, X. Y. Effect of Potassium on the yield of three sweet potato varieties. 1991. Asian Regional Vegetable Research Center. Proceedings of Training Workshop.
Daifu, M., Hongmin, L., DaPeng, L., and Yi, W. 2000. Sweetpotato varieties decline in China and the prevent practices. International Workshop on Sweetpotato Decline Study Sept. 8-9, 2000. Kyushu National Agricultural Experiment Station. Miyakonojo, Japan.

TABLE 2
A Compositional analysis of CX-1 variety

| | Test Values |
|---|---|
| Component | % of DM |
| Total Solids | 100 |
| Starch^{a} | 73.8 |
| Glucose, Maltose, Maltotriose^{b} | 0 |
| Protein | 2.45 |
| Fat | 1.86 |
| Ash (at 550° C.) | 2.89 |
| Remainder (fiber, non-starch | 19.0 |
| polysaccharides and lignin)^{c} | |

^{a}Starch content analyzed by AOAC Enzymatic analysis method Number (AOAC 979.1/AACC76.11)
^{b}Glucose, Maltose, and Maltotiose are estimated by HPLC PRAJ Standards method using Biorad Ion Exchange Column
^{c}Fiber and other organic matter is extrapolated by the difference in weight method
Woolfe, J. 1992. Sweetpotato an untapped food resource. Cambridge University Press, Cambridge, UK.

1. A horticultural method to maximize yield of high dry matter type sweetpotatoes, the method comprising:
(a) transplanting cuttings with a mechanical pocket transplanter into plant beds;
(b) watering the plants;
(c) planting a crop in the middle of a month with a climate essentially the same as average climate in mid-May in South Carolina;
(d) harvesting the crop at the last frost; and
(e) fertilizing the crop with nitrogen, phosphorus, potash, sulfur, boron, manganese and copper.

2. The method of claim 1 wherein the sweetpotatoes are from the variety CX-1.

3. The method of claim 1 wherein the cuttings are approximately 10 inches in length.

4. The method of claim 1 wherein the cuttings are transplanted approximately 12 inches apart in plant beds approximately 38 inches apart.

5. The method of claim 1 wherein the plants are watered by the transplanter and by natural rainfall.

6. The method of claim 1 wherein harvesting is at approximately 150-160 days after planting.

7. The method of claim 1 wherein fertilizing comprises approximately 45 pounds of nitrogen, approximately 110 pounds of phosphorus and potash, approximately 5 pounds of sulfur, approximately 0.4 pounds boron, approximately 10 pounds of manganese, and approximately 0.1-0.2 pounds of copper per acre.

8. A method to produce fuel ethanol, the method comprising:
(a) obtaining sweetpotato roots characterized by high dry matter; and
(b) producing fuel ethanol from the sweetpotato roots.

9. The method of claim 8 wherein the high dry matter is from about 28% to 40%.

10. The method of claim 8 wherein the sweetpotato roots are from the variety designated CX-1.

11. The method of claim 8 wherein the fuel ethanol is produced on a commercial scale.

12. A laboratory method to produce fuel ethanol, the method comprising:
(a) peeling, grating and drying high dry matter type sweetpotato roots;
(b) adding water to reconstitute the dried sweetpotato roots;
(c) adding amylase to the reconstituted roots and heating the results to boiling with stirring;
(d) cooling, adjusting pH to about 4.5-5.0;
(e) adding gluco-amylase, yeast and water; and
(f) incubating the roots after steps (a)-(e).

13. The method of claim 12 wherein the sweetpotato roots are from variety CX-1.

14. The method of claim 12 wherein the pH is adjusted with concentrated sulfuric acid.

15. The method of claim 12 wherein incubating is for approximately 42 hours.

16. A commercial method to produce fuel ethanol comprising:
(a) providing a slurry of sweetpotato roots;
(b) adding amylase and heating to boiling with stirring and boiling;
(c) cooling the slurry;
(d) adding gluco-amylase, yeast and water;
(e) incubating; and
(f) removing ethanol and water and separating the ethanol by fractional distillation.

17. The method of claim 16 wherein the slurry is produced by passing through a continuous double helix blender while adding a minimal amount of water.

18. The method of claim 16 wherein the slurry with amylase is boiled for about 90 minutes.

19. The method of claim 16 wherein the boiled slurry is then cooled to about 50° C. and the pH is adjusted by adding concentrated sulfuric acid.

20. The method of claim 15, further defined as incubated in a fermentation tank for about 36 hours.

21. The method of claim 16 further comprising removing final water from ethanol by molecular sieves.

22. A method to produce sweetpotato chips and fries, the method comprising:
(a) obtaining high dry matter sweet potatoes;
(b) washing, peeling and slicing the sweetpotatoes;
(c) for french fries cutting 30 mm circular slices into strips, for chips cutting 5 mm circular cross sections;
(d) pre-treating fries and chip slices in 15% saline for 10 minutes and drying the fries and chips; and
(e) cooking the cut pieces in hot oil until the chips or fries float and the water is cooked off.

23. A sweetpotato variety characterized by about 28-40% dry weight and about 70-80% starch content.

24. The sweetpotato of claim 23 is CX-1.

25. A sweetpotato produced by the method of claim 1.
